(19) United States
(12) Patent Application Publication
(10) Pub. No.: US 2026/0165762 A1
(43) Pub. Date: Jun. 18, 2026

(54) SURGICAL TOOL HANDLE WITH IMPROVED VISUALIZATION AND GRIP

(71) Applicant: The Board of Regents of the University of Oklahoma, Norman, OK (US)

(72) Inventors: Guilherme Barreiro, Oklahoma City, OK (US); Qinggong Tang, Norman, OK (US); Connor Matthew Young, Norman, OK (US); Kaustubh Ravindra Pandit, Oklahoma City, OK (US); Avery Ladymon, Chickasha, OK (US)

(21) Appl. No.: 19/418,655

(22) Filed: Dec. 12, 2025

Related U.S. Application Data
(60) Provisional application No. 63/733,320, filed on Dec. 12, 2024.

(51) Int. Cl. A61B 18/00; A61B 17/00; A61B 90/30

(52) U.S. Cl. CPC A61B 18/00; A61B 90/30; A61B 2017/00424; A61B 2017/00429; A61B 2017/00464; A61B 2017/00477; A61B 2018/00178; A61B 2018/00601; A61B 2018/0091; A61B 2090/309

(57) ABSTRACT

Surgical tool handles, including a surgical tool handle having a tail portion, a tool connection end, and a grip portion extending between the tail portion and the tool connection end; wherein the tool connection end is configured to engage with an end of a surgical tool; the grip portion has a first sidewall and a second sidewall and a grip width extending between the first sidewall and the second sidewall; the grip portion has a top surface and a bottom surface and a grip height extending between the top surface and the bottom surface; the first sidewall and the second sidewall of the grip portion define a viewing aperture through the top surface, the grip height, and the bottom surface, the viewing aperture positioned proximate to the tool connection end; and the surgical tool handle has a longitudinal axis and the viewing aperture is positioned on the longitudinal axis.

### CROSS-REFERENCE TO RELATED APPLICATIONS

This application claims the benefit under 35 U.S.C. § 119(e) of the U.S. Provisional Application identified by Ser. No. 63/733,320, titled “SURGICAL TOOL HANDLE WITH IMPROVED VISUALIZATION AND GRIP”, filed Dec. 12, 2024, the entire contents of which are hereby expressly incorporated by reference herein.

### GOVERNMENT SUPPORT

Not Applicable

### BACKGROUND

Scalpel-type implements have been used in surgical procedures for millennia in one form or another. In the late 1800s, Schott patented the idea of attaching a disposable blade to the scalpel handle, allowing for sharp blades and sterile fields. In 1915, the Bard-Parker Company patented the colloquial “B.P. handle” for scalpels, which is still used today with over thirty blade varieties adapted to fit the handle.

Since that time, little innovation has occurred in the field of scalpels, although they remain a staple instrument for medical, veterinary, and dental professionals. Current scalpel handles are either disposable or reusable upon sterilization, and available in many different sizes and materials. The use of surgical blades has also been paired with new methods of cutting tissue, such as lasers and electrical devices that allow for precise cuts with limited blood loss.

Conventional scalpels and electrosurgical tools have solid, linear, flat or pen-like handles. Such designs force an awkward, fatigue-inducing grip that is unconducive to blade control and impedes the view of any surgical markings. As a result, medical professionals, inexperienced surgeons in particular, may inadvertently tilt cutting instruments in an effort to better see what they are cutting or as a result of fatigue.

However, incision angle is an important factor in the wound healing process and resulting scar tissue formation. In many fields, especially cosmetic surgery, the precise incision technique can directly correlate to patient healing and satisfaction. In turn, there is an ongoing need for improved surgical tool handles that facilitate controlled, unobstructed surgical procedures.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function. In the drawings:

FIG. 1 is a top view of one embodiment of an exemplary surgical tool handle in accordance with the present disclosure.

FIG. 2 is a perspective side view of the surgical tool handle embodiment of FIG. 1.

FIG. 3 is a perspective view of another embodiment of an exemplary surgical tool handle in accordance with the present disclosure.

FIG. 4 is a second side view of the surgical tool handle embodiment of FIG. 3.

FIG. 5 is a top view of the surgical tool handle embodiment of FIG. 3.

FIG. 6 is a bottom view of the surgical tool handle embodiment of FIG. 3.

FIG. 7 illustrates use of the surgical tool handle embodiment of FIG. 3 in accordance with the present disclosure.

FIG. 8 is a perspective hand-held view of the surgical tool handle embodiment of FIG. 3, illustrating the viewing aperture.

FIG. 9 is a bottom perspective view of an exemplary surgical tool handle embodiment with a light source, in accordance with the present disclosure.

FIG. 10 is a side perspective view of an exemplary surgical tool handle embodiment with a laser source, in accordance with the present disclosure.

FIG. 11 is a side perspective view of an exemplary surgical tool handle embodiment in conjunction with an electrosurgical tool, in accordance with the present disclosure.

FIG. 12 is a first end view of the surgical tool handle embodiment of FIG. 3.

FIG. 13 is a second end view of the surgical tool handle embodiment of FIG. 3.

### DETAILED DESCRIPTION

The following detailed description refers to the accompanying drawings. The same reference numbers in different drawings may identify the same or similar elements.

The mechanisms proposed in this disclosure circumvent the problems described above. The present disclosure describes surgical tool handles and surgical instruments. An exemplary embodiment includes a surgical tool handle having a tail portion, a tool connection end, and a grip portion extending between the tail portion and the tool connection end; wherein the tool connection end is configured to engage with an end of a surgical tool; wherein the grip portion has a first sidewall and a second sidewall and a grip width extending between the first sidewall and the second sidewall; wherein the grip portion has a top surface and a bottom surface and a grip height extending between the top surface and the bottom surface; wherein the first sidewall and the second sidewall of the grip portion define a viewing aperture through the top surface, the grip height, and the bottom surface, the viewing aperture positioned proximate to the tool connection end; and wherein the surgical tool handle has a longitudinal axis and the viewing aperture is positioned on the longitudinal axis.

Before further describing various embodiments of the apparatus, component parts, and methods of the present disclosure in more detail by way of exemplary description, examples, and results, it is to be understood that the embodiments of the present disclosure are not limited in application to the details of apparatus, component parts, and methods as set forth in the following description. The embodiments of the apparatus, component parts, and methods of the present disclosure are capable of being practiced or carried out in various ways not explicitly described herein. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description. While the apparatus, component parts, and methods of the present disclosure have been described in terms of particular embodiments, it will be apparent to those of skill in the art that variations may be applied to the apparatus, component parts, and/or methods and in the steps or in the sequence of steps of the method described herein without departing from the concept, spirit, and scope of the inventive concepts as described herein. All such similar substitutes and modifications apparent to those having ordinary skill in the art are deemed to be within the spirit and scope of the inventive concepts as disclosed herein.

All patents, published patent applications, and non-patent publications referenced or mentioned in any portion of the present specification are indicative of the level of skill of those skilled in the art to which the present disclosure pertains, and are hereby expressly incorporated by reference in their entirety to the same extent as if the contents of each individual patent or publication was specifically and individually incorporated herein.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms and phrases, unless otherwise indicated, shall be understood to have the following meanings: The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The phrase “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, observer error, and combinations thereof, for example. The term “about” or “approximately”, where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 80% of the time, at least 90% of the time, at least 91% of the time, at least 92% of the time, at least 93% of the time, at least 94% of the time, at least 95% of the time, at least 96% of the time, at least 97% of the time, at least 98% of the time, or at least 99% of the time.

Where used herein, the pronoun “we” is intended to refer to all persons involved in a particular aspect of the investigation disclosed herein and as such may include non-inventor laboratory assistants and non-inventor collaborators working under the supervision of the inventor(s).

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, a range of 1-1,000 includes, for example, 1-10, 10-20, 20-30, 30-40, 40-50, 50-60, 60-75, 75-100, 100-150, 150-200, 200-250, 250-300, 300-400, 400-500, 500-750, 750-1,000, and includes ranges of 1-20, 10-50, 50-100, 100-500, and 500-1,000. The range 100 units to 2000 units therefore refers to and includes all values or ranges of values of the units, and fractions of the values of the units and integers within said range, including for example, but not limited to 100 units to 1000 units, 100 units to 500 units, 200 units to 1000 units, 300 units to 1500 units, 400 units to 2000 units, 500 units to 2000 units, 500 units to 1000 units, 250 units to 1750 units, 250 units to 1200 units, 750 units to 2000 units, 150 units to 1500 units, 100 units to 1250 units, and 800 units to 1200 units. Any two values within the range of about 100 units to about 2000 units therefore can be used to set the lower and upper boundaries of a range in accordance with the embodiments of the present disclosure. More particularly, a range of 10-12 units includes, for example, 10, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, and 12.0, and all values or ranges of values of the units, and fractions of the values of the units and integers within said range, and ranges which combine the values of the boundaries of different ranges within the series, e.g., 10.1 to 11.5.

The use of ordinal number terminology (i.e., “first”, “second”, “third”, “fourth”, etc.) is solely for the purpose of differentiating between two or more items and, unless explicitly stated otherwise, is not meant to imply any sequence or order or importance to one item over another or any order of addition.

In accordance with the present disclosure, a surgical tool handle with an open geometry viewfinder can be adapted to many surgical instruments. Particularly, the present disclosure may advantage the user of a tissue cutting tool, including, but not limited to, scalpels, electrocautery instruments, and surgical lasers. The disclosed handle allows for direct visualization of the surgical field, thus addressing the long-standing deficiencies in surgical tool design mentioned above. Further, embodiments of the surgical tool handle include ergonomically positioned finger grip(s) for comfortable gripping and handling, which improves performance and efficiency.

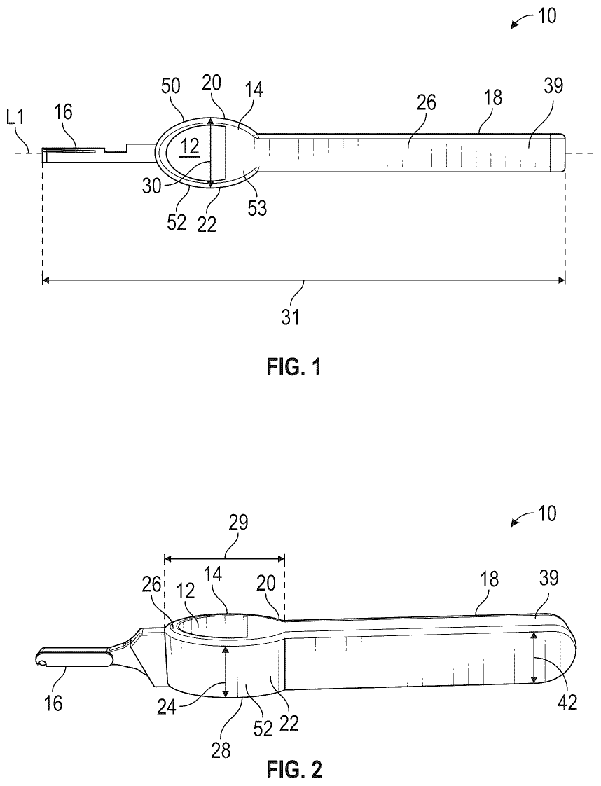

Referring now to the drawings, in particular to FIGS. 1 and 2, a surgical tool handle 10 (which may be referred to herein as simply handle 10) having a viewing aperture 12 is illustrated. The handle 10 has a tail portion 18, a tool connection end 16, and a grip portion 14 that extends between the tool connection end 16 and the tail portion 18. The surgical tool handle 10 has a handle length 31 defined along a longitudinal axis L1. In some embodiments, the longitudinal axis L1 is along a midline of the surgical tool handle 10 along the handle length 31.

The surgical tool handle length 31 may vary accordingly depending on the intended use of the surgical tool handle 10 and user preferences. The handle length 31 may be generally between 140 millimeters to 215 millimeters. In some embodiments, the handle length 31 is between 165 millimeters and 175 millimeters. In some embodiments, the handle length 31 is approximately 170 millimeters.

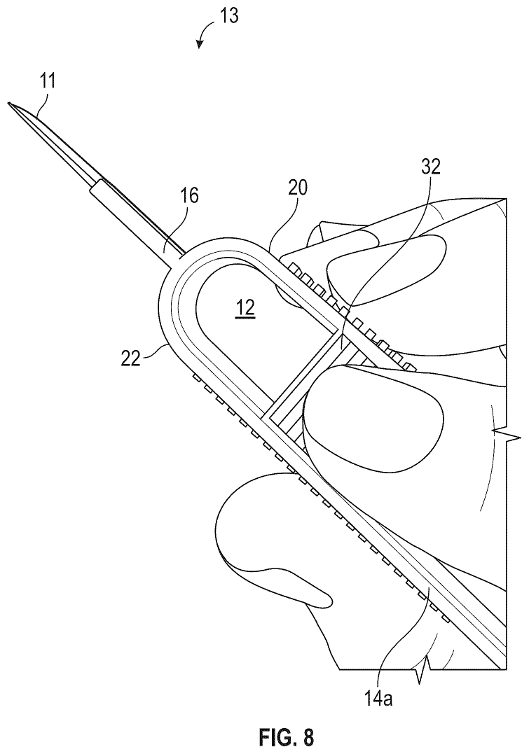

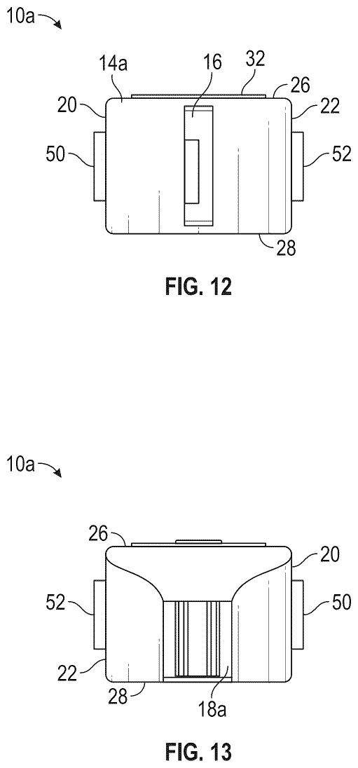

The tool connection end 16 of the handle 10 may be configured to engage with a surgical tool 11. The handle 10 in conjunction with the surgical tool 11 may be referred to as a surgical instrument 13 (FIGS. 8, 10, 12). In some embodiments, the surgical tool 11 may be a scalpel blade. In some embodiments, the surgical tool 11 may be one or more of a blade, a needle, a tip, a fiber, other end effectuator, or other type of tool used in surgery or for medical procedures.

The grip portion 14 of the handle 10 has a first sidewall 20, a second sidewall 22, and a grip width 30 extending between and across the first sidewall 20 and the second sidewall 22. The grip portion 14 further has a top surface 26, a bottom surface 28, and a grip height 24 extending between the top surface 26 and the bottom surface 28. The grip portion 14 has a grip length 29 extending between the tool connection end 16 and the tail portion 18.

One or more sections of the top surface 26 and/or the bottom surface 28 of the grip portion 14 may have one or more textures 53. For example, the texture may be one or more of ridges, granulations, raised portions, protrusions, and indentations.

The first sidewall 20 and the second sidewall 22 of the grip portion 14 define the viewing aperture 12 between the first sidewall 20 and the second sidewall 22. The viewing aperture 12 is positioned proximate to the tool connection end 16 of the handle 10. The viewing aperture 12 spans the grip height 24, extending through the top surface 26 and the bottom surface 28 of the grip portion 14.

In some embodiments, the viewing aperture 12 may be an open space. In some embodiments, the viewing aperture 12 may comprise a transparent material.

In some embodiments, the viewing aperture 12 may have a shape that is at least partially rounded.

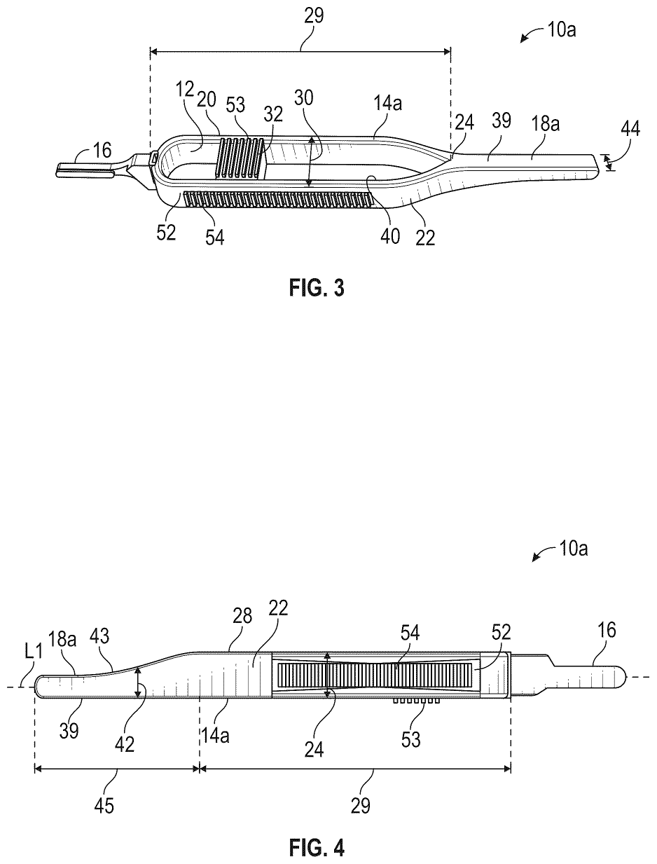

The first sidewall 20 of the grip portion 14 has a first outside surface 50 and the second sidewall 22 of the grip portion 14 has a second outside surface 52. In some embodiments, one or more portions of the first outside surface 50 of the first sidewall 20 and/or the second outside surface of the second sidewall 22 may have a texture 54 configured for gripping (see FIG. 3). For example, the texture may be one or more of ridges, granulations, raised portions, protrusions, and indentations.

The height of the grip height 24 of the grip portion 14 of the handle 10 may be configured for ergonomic gripping of the handle 10. The height of the grip height 24 may be configured for a user to grasp the grip portion 14 of the handle 10. In some embodiments, the height of the grip height 24 may be between five millimeters and fifteen millimeters. In some embodiments, the height of the grip height 24 may be between ten millimeters and eleven millimeters.

In some embodiments, the grip height 24 may vary in height along the grip length 29. For example, in one embodiment, the grip height may vary in height between ten millimeters and eleven millimeters along the grip length 29.

The grip portion 14 of the handle 10 has a grip width 30, extending between and across the first sidewall 20 and the second sidewall 22. The grip width 30 is wider than conventional scalpel or electrocautery tool handles. The grip width 30 is designed ergonomically to reduce fatigue or tension of a user, and also to accommodate the viewing aperture 12 of the grip portion 14. In some embodiments, the grip width 30 is between ten millimeters and thirty millimeters. In some embodiments, the grip width is nineteen millimeters.

In some embodiments, different portions of the grip portion 14 may also be different widths.

In one embodiment, the grip width 30 of the grip portion 14 narrows in a transition into the tail portion 18. In one embodiment, the grip width 30 of the grip portion 14 narrows in a transition into the tool connection end 16. Of course, it will be understood that the tail portion 18 and/or the tool connection end 16 may have other shapes than that shown in transitions into the grip portion 14.

The viewing aperture 12 enhances visibility of the surgical field, such as along the longitudinal axis L1 of the surgical tool.

When the handle 10 is in use, the aperture 12 in the handle 10 is configured to give a user holding the surgical tool handle 10 and looking through the aperture 12 of the surgical tool handle 10 an unobstructed view of the tissue to be cut or treat, without having to tilt the hand of the user which tilts the surgical tool handle 10 including the surgical tool 11, and without interrupting the procedure to check for accuracy of position. For example, a user can make an incision without unintentionally tilting the surgical tool 11 or cutting a patient in an unintended area or direction (off target) because the user can see the immediate surgical field (such as, for example, pre-operative drawing lines) through the viewing aperture 12 of the surgical tool handle 10 while cutting.

In some embodiments, the viewing aperture 12 may have a length extending between 5% and 60% of the surgical tool handle length 31 along the longitudinal axis L1 of the handle 10. In some embodiments, the viewing aperture 12 may have a length extending between 5% and 15% of the surgical tool handle length 31 along the longitudinal axis L1 of the handle 10. In some embodiments, the viewing aperture 12 may have a length between seven and thirty-two millimeters. In some embodiments, the viewing aperture 12 may have a length between fifteen millimeters and twenty millimeters.

FIGS. 3-10, 12, and 13 illustrate another embodiment of a surgical tool handle 10a, with is similar to the surgical tool handle 10 previously described, except as described below. In the surgical tool handle 10a, a grip portion 14a may have a finger-support bridge 32 (hereinafter support bridge 32) positioned within a half of the grip portion 14a proximate to the tool connection end 16. The support bridge 32 further improves precision by providing an ergonomic placement for a finger of a user (such as an index finger). The support bridge 32 may extend between the first sidewall 20 and the second sidewall 22 of the grip portion 14a, connecting the first sidewall 20 and the second sidewall 22.

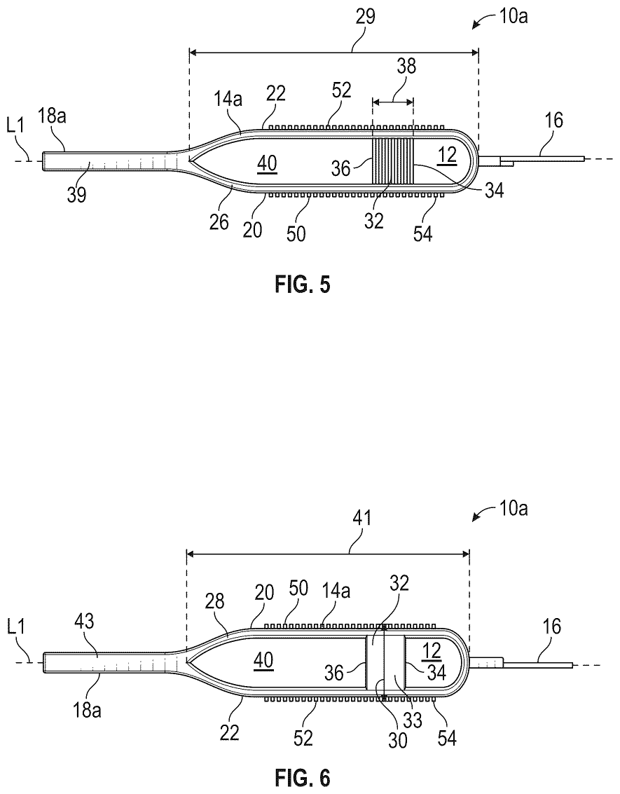

The support bridge 32 may have a tool side 34 proximate to the tool connection end 16 and a tail side 36 proximate to the tail portion 18 (FIG. 5). The support bridge 32 may have a bridge length 38 extending between the tool side 34 and the tail side 36 of the support bridge 32.

The bridge length 38 of the support bridge 32 of the grip portion 14 may be sized to generally accommodate a size of a fingertip of a typical user. For example, the support bridge length 38 may be between ten millimeters and twenty millimeters. In some embodiments, the support bridge length 38 of the support bridge 32 may be between thirteen millimeters and fourteen millimeters. In some embodiments, the support bridge length 38 of the support bridge 32 may be 5% to 13% of the handle length 31.

The one or more sections of the top surface 26 and/or the bottom surface 28 of the grip portion 14a that may have the one or more textures 53 may include one or more sections of the support bridge 32.

In some embodiments, the tool side 34 of the support bridge 32 in conjunction with the first sidewall 20 and the second sidewall 22 of the grip portion 14a define the viewing aperture 12.

As illustrated in FIGS. 3-6, in some embodiments, the first sidewall 20, the second sidewall 22, and the tail side 36 of the support bridge 32 of the grip portion 14a cooperate to form a second viewing aperture 40 (which may be referred to herein as second aperture 40). The second viewing aperture 40 may extend through the top surface 26, grip height 24, and bottom surface 28 of the grip portion 14a. The second viewing aperture 40 has a second length 41 along the longitudinal axis L1 of the handle 10.

In some embodiments, the second length 41 of the second viewing aperture 40 of the grip portion 14 may be generally one third of the handle length 31. In some embodiments, the second length 41 of the second viewing aperture 40 may be between forty millimeters and seventy millimeters. In some embodiments, the second length 41 of the second viewing aperture 40 may be between fifty-five millimeters and sixty millimeters. For example, in one embodiment the second length 41 of the second aperture 40 is fifty-seven millimeters.

In some embodiments which include the viewing aperture 12 and the second viewing aperture 40, the support bridge 32 may have a length of between ten millimeters and fifteen millimeters. In some embodiments which include the viewing aperture 12 and the second viewing aperture 40, the support bridge 32 may have a length of approximately thirteen millimeters.

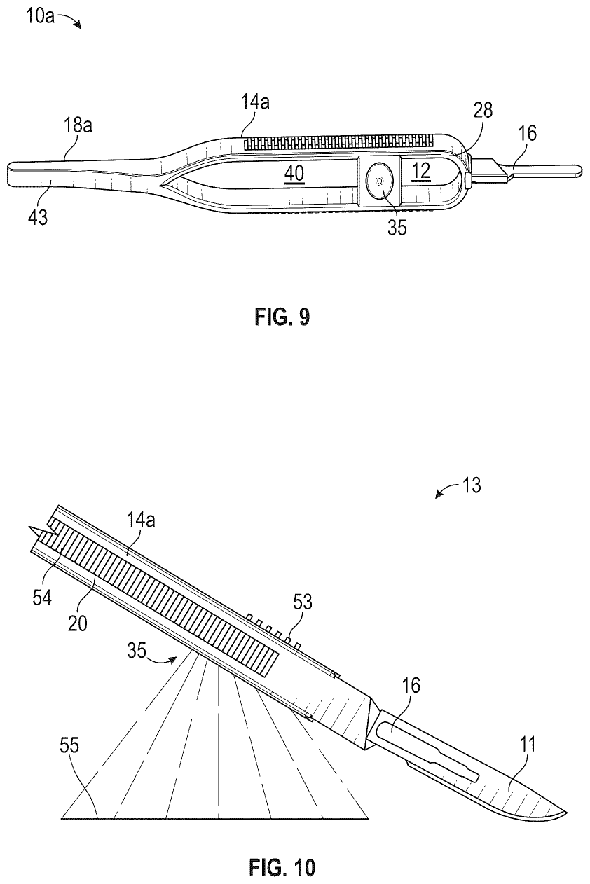

Now referring to FIGS. 9 and 10, the handle 10, 10a may comprise a light source 35. The light source 35 may be positioned on and/or in the handle 10, 10a such that light from the light source 35 further enhances surgical field visibility for the instrument 13. The light source 35 may be positioned in the handle 10, 10a so as to illuminate a local surgical working area.

In some embodiments, the light source 35 is positioned in the grip portion 14, 14a of the handle 10, 10a, such as on, and/or in, the bottom surface 28 of the grip portion 14, 14a. In some embodiments, the light source 35 may be positioned on, and/or in, the support bridge 32 of the grip portion 14a.

The light source 35 may comprise a LED light. The light source 35 may comprise a laser or similarly focused light source that illuminates concentrated areas. The light source 35 may provide a diffuse light and/or may provide a concentrated light, such as a projected line 55. In one embodiment, the concentrated light may be shaped to follow the longitudinal axis L1 of the handle 10, 10a, and/or of the surgical tool 11, and projected onto a cutting surface, such as shown as a fixed beam in the projected line 55 in FIG. 10, that the user can reference for alignment and/or follow with the surgical tool 11. The light source 35 may provide light that is visible through one or more of the viewing aperture 12 and the second viewing aperture 40.

The light source 35 may comprise any light source appropriate for surgical applications, such as one or more light bulbs, light emitting diodes (LEDs), fluorescent tubes, or digital lasers. The light source 35 may be powered by any source known in the art, such as batteries, optical fibers, and/or an electrical connection. Further, it is to be understood that the handle 10, 10a may also include control mechanisms, such as buttons or switches, for operating the light source 35.

Returning now to FIGS. 1-6, a tail portion 18, 18a is positioned in the handle 10, 10a opposite from the tool connection end 16 along the handle length 31 of the handle 10, 10a. The tail portion 18, 18a has a top surface 39, a bottom surface 43, a tail height 42 extending between the top surface 39 and the bottom surface 43, a tail width 44, and a tail length 45 along the longitudinal axis L1 of the handle 10.

The tail length 45 may be generally 25% to 30% of the handle length 31. The tail length 45 may be 27% of the handle length 31. In some embodiments, the tail length 45 is between thirty-seven millimeters and fifty-one millimeters. In some embodiments, the tail length 45 is approximately forty-seven millimeters.

The tail height 42, extending between the top surface 39 and bottom surface 43, may be between three millimeters and eleven millimeters. In some embodiments, the tail height 42 is between five and ten millimeters.

In some embodiments, the tail width 44 may be between three and twelve millimeters. In some embodiments, the tail width 44 is between five and eleven millimeters. In some embodiments, the tail width 44 is approximately ten millimeters.

In some embodiments, at least a portion of the tail width 44 and at least a portion of the tail height 42 may be the same as, or have the same range as, the grip width 30 and the grip height 24, respectively. For example, in some embodiments, a transition between the tail portion 18 and a grip portion 14a of the handle 10a may be indistinguishable, as is shown in FIGS. 1 and 2. In some embodiments, as illustrated in FIGS. 3-6, the transition from the grip portion 14a to the tail portion 18a may include a tapering of the grip portion 14 such that the tail height 42 and the tail width 44 are less than the grip height 24 and/or the grip width 30, respectively.

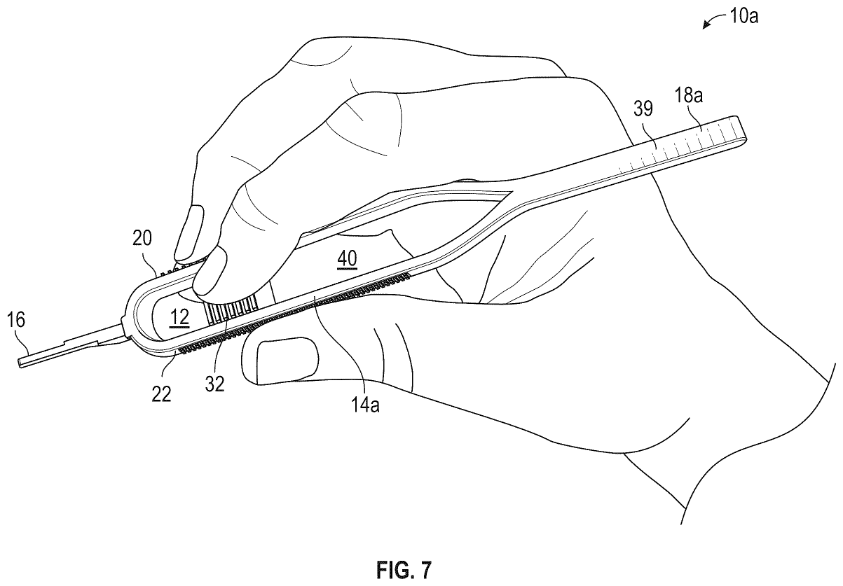

In some embodiments, the tail portion 18, 18a may be ergonomically shaped to accommodate a curvature of a typical hand of a user during use of the tool. In some embodiments, the ergonomic shape of the tail portion 18, 18a may be specific to the surgical tool 11 and how the surgical tool handle 10, 10a is commonly held by the user. In some embodiments, as is illustrated in FIGS. 4 and 7, the tail portion 18a may be curved between the top surface 39 and the bottom surface 43 of the tail portion 18a.

In some embodiments, the tail height 42 at a proximal end of the tail portion 18a that is proximate to the grip portion 14a may be greater than the tail height 42 at a distal end of the tail portion 18a that extends away from the grip portion 14a along the length 31 of the handle 10, and the tail length 45 of the tail portion 18a may transition from the tail height 42 at the proximal end of the tail portion 18a to the lesser tail height 42 at the distal end of the tail portion 18a in a slope or a curve. In some embodiments, the top surface 39 of the tail portion 18a may be in the same plane as the top surface 26 of the grip portion 14a and the bottom surface 43 of the tail portion 18a may be curved, such that the bottom surface 43 is concave. The curve b may minimize friction and discomfort during use. In some embodiments, the shape of the curve may be for aesthetic reasons and may not be functional.

As shown in FIG. 10, the tool connection end 16 is configured to engage with the surgical tool 11, such as a surgical blade, or with other types of surgical tools 11. In some embodiments, a connection between the surgical tool 11 and the handle 10, 10a may be a conventional connection, such as typically used for connecting blades with conventional scalpel handles. For example, the tool connection end 16 may have one or more protrusions configured to engage with an opening within a surgical blade. Conversely, in other embodiments, the tool connection end 16 may have one or more openings configured to engage with one or more protrusions of a surgical tool 11.

In some embodiments, the connection between the surgical tool 11 and the handle 10, 10a may be designed specifically for a particular surgical tool 11.

In some embodiments, the handle 10, 10a may be used as a non-disposable tool and may have any conventional fastening means or adopt the suitable sizes and shapes known in the art to accommodate conventional and specialized end effectuators.

The connection between the surgical tool 11 and the handle 10, 10a may be a connection that is engageable and disengageable without damaging the surgical tool 11 or the handle 10, 10a.

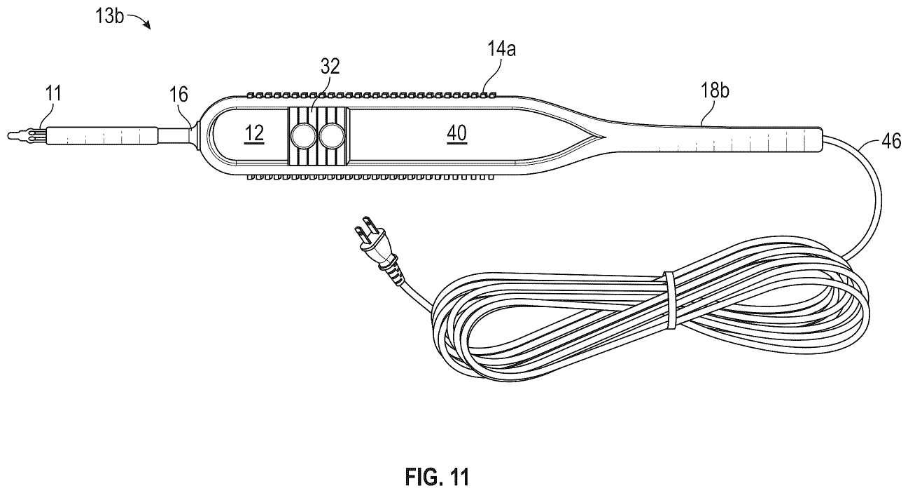

However, the connection between the surgical tool 11 and the handle 10, 10a may be permanent, such that the surgical tool 11 is not removable from the handle 10, 10a without damaging the surgical tool 11 or the handle 10, 10a. This may be the case for instruments where the handle 10, 10a and the surgical tool 11 are manufactured as one unit, such as, for example, disposable and/or multi-use units. For example, as shown in FIG. 11, the surgical tool 11 may be an electrocautery tip that may be fixed to a handle 10b in an embodiment of an electrocautery surgical instrument 13b.

Power supply is often necessary for modern tools that cut tissue mechanically, cut tissue with energy, and coagulate tissue. In such embodiments as shown in FIG. 11 an instrument 13b may have a handle 10b that may comprise and/or may be used in conjunction with, a power supply, electrodes, and/or user controls. To accommodate a surgical tool 11 that is an electrosurgical tool, the handle 10, 10a, 10b may be configured, mechanically and electrically, with an electrical connection 46. As exemplified in FIG. 11, the handle 10b may have a tail portion 18b that may be configured to receive an electrical connection 46 (such as a cord) that supplies power to the cautery tip at the tool connection end 16.

The embodiments disclosed herein may be reusable or disposable, and thus may be manufactured from variety of materials. In some embodiments, the handle 10, 10a, 10b may be a single piece, which may be manufactured using an injection molding process. Thus, a moldable plastic material may be used for disposable, single-use tools. In some embodiments, reusable handles 10, 10a, 10b may be made from materials that can be sterilized, such as metals like titanium, steel, aluminum, gold, and silver. Other materials that can be sterilized in a hospital may be used as well, such as glass, ceramics, silicones, and heat-resistant plastics/urethanes like polypropylene, polysulfone, and polytetrafluoroethylene. In some embodiments, the handle 10, 10a, 10b may be made of a combination of materials, such as those listed above.

Conventionally, surgical tool handles are non-ergonomic, causing fatigue to the user, and often obstruct direct viewing of the surgical field during use. In accordance with the present disclosure, a surgical tool handle with a viewing aperture and wider grip design allows a user to see the working area and precisely cut tissue.

An example of the surgical tool handle 10, 10a in use hypothetical will now be described.

Operation of the handle 10, 10a may be accomplished by a user placing the fingertip of the index finger on the top surface 26 of the grip portion 14a, particularly on the support bridge 32, and then placing the middle finger and thumb on the first sidewall 20 and second sidewall 22 of the grip portion, respectively, to support the grip. The user gripping the handle 10,10a with their fingers may also adjust where the grip portion 14a and tail portion 18a contact the hand to suite their preferred grip style. For example, as shown in FIG. 7, a user gripping the handle 10,10a in a pencil/pen-style grip will position the concave bottom surface 43 of the tail portion 18a against the curvature of the proximal metacarpal interphalangeal joint of the user, which may stabilize the transition between the grip portion 14a and tail portion 18a along the palmar digital crease of the index finger.

A user gripping the handle 10, 10a may then guide the attached surgical tool 11 to the surgical field and locate the precise tissue to be cut, placing the tool 11 on the location. Using the index finger to guide and provide pressure to the surgical tool 11, as is standard technique in using instruments such as a scalpel, the user may begin cutting tissue. Simultaneously, the user may view the area they are about to cut by looking through the viewing aperture 12. Viewing the localized tissue to be cut and any corresponding markings while operating the handle 10, 10a allows the user to orient and guide the surgical tool 11 without any disruption to the flow or angles of movement.

The foregoing description provides illustration and description, but is not intended to be exhaustive or to limit the inventive concepts to the precise form disclosed. Modifications and variations are possible in light of the above teachings or may be acquired from practice of the methodologies set forth in the present disclosure.

Even though particular combinations of features are recited in the claims and/or disclosed in the specification, these combinations are not intended to limit the disclosure. In fact, many of these features may be combined in ways not specifically recited in the claims and/or disclosed in the specification. Although each dependent claim listed below may directly depend on only one other claim, the disclosure includes each dependent claim in combination with every other claim in the claim set.

No element, act, or instruction used in the present application should be construed as critical or essential to the invention unless explicitly described as such outside of the preferred embodiment. Further, the phrase “based on” is intended to mean “based, at least in part, on” unless explicitly stated otherwise.

What is claimed is:

1. A surgical tool handle having a tail portion, a tool connection end, and a grip portion extending between the tail portion and the tool connection end;
wherein the tool connection end is configured to engage with an end of a surgical tool;
wherein the grip portion has a first sidewall and a second sidewall and a grip width extending between the first sidewall and the second sidewall;
wherein the grip portion has a top surface and a bottom surface and a grip height extending between the top surface and the bottom surface;
wherein the first sidewall and the second sidewall of the grip portion define a viewing aperture through the top surface, the grip height, and the bottom surface, the viewing aperture positioned proximate to the tool connection end; and
wherein the surgical tool handle has length defined along a longitudinal axis and the viewing aperture is positioned on the longitudinal axis.

2. The surgical tool handle of claim 1, wherein the grip width is in a range of between ten millimeters and thirty millimeters.

3. The surgical tool handle of claim 1, wherein the first sidewall and the second sidewall of the grip portion are connected by a finger-support bridge, the finger-support bridge having a tool side, wherein the first sidewall, the second sidewall, and the tool side of the finger-support bridge define the viewing aperture.

4. The surgical tool handle of claim 1, wherein the surgical tool handle has a length and wherein the viewing aperture extends between 5% and 60% of the length of the surgical tool handle.

5. The surgical tool handle of claim 1, wherein the tail portion has a tail height and wherein the grip height of the grip portion is greater than the tail height; and wherein the tail portion has a tail width and, wherein the grip width of the grip portion is wider than the tail width.

6. The surgical tool handle of claim 5, wherein the tail portion has a tail height that slopes from the grip height to a height less than the grip height.

7. The surgical tool handle of claim 1, wherein the viewing aperture is a first viewing aperture;
wherein the first sidewall and the second sidewall of the grip portion define a second viewing aperture through the top surface, the grip height, and the bottom surface; and
wherein the second viewing aperture is positioned proximate to the tail portion.

8. The surgical tool handle of claim 1, wherein the viewing aperture is a first viewing aperture; wherein the first sidewall and the second sidewall of the grip portion are connected by a finger-support bridge, the finger-support bridge having a tool side and a tail side; wherein the first sidewall of the grip portion, the second sidewall of the grip portion, and the tail side of the finger-support bridge of the grip portion define a second viewing aperture through the top surface, the grip height, and the bottom surface of the grip portion; and wherein the second viewing aperture is positioned proximate to the tail portion.

9. The surgical tool handle of claim 8, wherein the first viewing aperture is smaller than the second viewing aperture.

10. The surgical tool handle of claim 7, wherein the first viewing aperture and the second viewing aperture are separated by a finger-support bridge.

11. The surgical tool handle of claim 10, wherein the finger-support bridge has an tool side and a tail side;
wherein the first sidewall, the second sidewall, and the tool side of the finger-support bridge define the first viewing aperture; and
wherein the first sidewall, the second sidewall and the tail side of the finger-support bridge define the second viewing aperture.

12. The surgical tool handle of claim 10, wherein the finger-support bridge has an under surface, the surgical tool handle comprising a light source positioned in the under surface of the finger-support bridge.

13. The surgical tool handle of claim 1, wherein the tool connection end has a protrusion configured to engage a first portion of the end of a surgical tool.

14. The surgical tool handle of claim 1, wherein the surgical tool is a blade, and wherein the tool connection end has a slot configured to receive a first portion of the end of a blade and has an extension configured to engage with a second portion of the end of the blade.

15. The surgical tool handle of claim 1, wherein the viewing aperture is at least partially rounded.

16. The surgical tool handle of claim 1, wherein at least a portion of the top surface of the grip portion is textured.

17. The surgical tool handle of claim 1, wherein at least a portion of the first sidewall and at least a portion of the second sidewall is textured with ridges.

18. The surgical tool handle of claim 1, comprising a light source positioned within the grip portion, the light source configured to generate light out of the under surface of the grip portion.

19. The surgical tool handle of claim 1, comprising a laser light positioned within the grip portion, the laser light configured to generate a focused light beam out of the bottom surface of the grip portion.

20. The surgical tool handle of claim 1, wherein the tool connection end is configured to engage with an end of an electrosurgical tool, and wherein the surgical tool handle comprises an electrical connection configured to provide electrical power to the electrosurgical tool when engaged with the tool connection end.
